(19) United States
(12) Patent Application Publication
(10) Pub. No.: US 2020/0139100 A1
(43) Pub. Date: May 7, 2020

(54) MEDICAL PORT LOCATOR

(71) Applicants: Trinity Health Corporation, Livonia, MI (US); Grand Valley State University, Allendale, MI (US)

(72) Inventors: Charles A. Hyde, Cedar Springs, MI (US); Brad Wassink, Grand Rapids, MI (US); Cody Teliczan, Jenison, MI (US); Robin Petersen, Grand Rapids, MI (US); Justin Phillips, Allendale, MI (US); John Farris, Grand Rapids, MI (US)

(21) Appl. No.: 16/676,621

(22) Filed: Nov. 7, 2019

Related U.S. Application Data
(60) Provisional application No. 62/756,634, filed on Nov. 7, 2018.

(51) Int. Cl. A61M 39/02

(52) U.S. Cl. CPC A61M 39/0247; A61M 2039/0205; A61M 2039/0238; A61M 39/0208

(57) ABSTRACT

A medical port locator includes a front side and a rear side. In embodiments, the front side includes a receiving/recessed portion and an upper portion. The receiving/recessed portion may be configured to engage at least a portion of a separate medical device, such as a medical port. In embodiments, the medical port locator has a first portion that may be configured to cover a portion of at least one finger of a user and a second portion that may be configured to cover a portion of at least another finger of a user.

### CROSS-REFERENCE TO RELATED APPLICATIONS

This application claims the benefit of U.S. Provisional Patent Application Ser. No. 62/756,634, filed on Nov. 7, 2018, the disclosures of which is hereby incorporated herein by reference in its entirety.

### TECHNICAL FIELD

The present disclosure generally relates to a device that can, among other things, protect a user's hand, and/or facilitates the locating and/or gripping of a device, such as a medical port.

### BACKGROUND

This background description is set forth below for the purpose of providing context only. Therefore, any aspect of this background description, to the extent that it does not otherwise qualify as prior art, is neither expressly nor impliedly admitted as prior art against the instant disclosure.

Medical devices, including venous access devices, or medical ports, may be surgically implanted beneath the skin of a patient. Such medical ports can, among other things, provide easier insertion of devices and/or delivery of medicines. For example, some medical ports may be used in connection with catheters or other port-related medical devices. Medical ports may be used, for example, to administer medications or to facilitate blood draws.

While medical ports can provide a measure of convenience, it can be desirable to provide a device that can help medical professionals use such ports.

As such, there is a desire for solutions/options that improve the use of medical ports. The foregoing discussion is intended only to illustrate examples of the present field and should not be taken as a disavowal of scope.

### SUMMARY

A medical port locator includes a front side and a rear side. In embodiments, the front side includes a receiving/recessed portion and an upper portion. The receiving/recessed portion may be configured to engage at least a portion of a separate medical device, such as a medical port. In embodiments, rear side is configured to receive at least a portion of a user's hand. With embodiments, the medical port locator includes a first portion that may be configured to cover a portion of at least one finger of a user and a second portion that may be configured to cover a portion of at least another finger of a user.

The foregoing and other aspects, features, details, utilities, and/or advantages of embodiments of the present disclosure will be apparent from reading the following description, and from reviewing the accompanying drawings.

### BRIEF DESCRIPTION OF THE DRAWINGS

FIG. 1 is a front-top-side perspective view of an embodiment of a medical port locator, along with a medical port and other related components according to teachings of the present disclosure.

FIG. 2 is a rear-top-side perspective view of an embodiment of a medical port locator, along with a medical port and other related components according to teachings of the present disclosure.

FIG. 3 is a front-top-side perspective view of an embodiment of a medical port locator.

### DETAILED DESCRIPTION

Reference will now be made in detail to embodiments of the present disclosure, examples of which are described herein and illustrated in the accompanying drawings. While the present disclosure will be described in conjunction with embodiments and/or examples, it will be understood that they are not intended to limit the present disclosure to these embodiments and/or examples. On the contrary, the present disclosure is intended to cover alternatives, modifications, and equivalents.

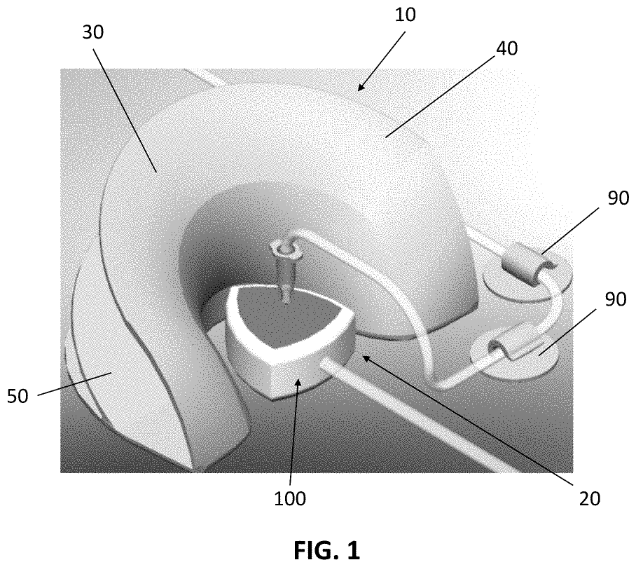

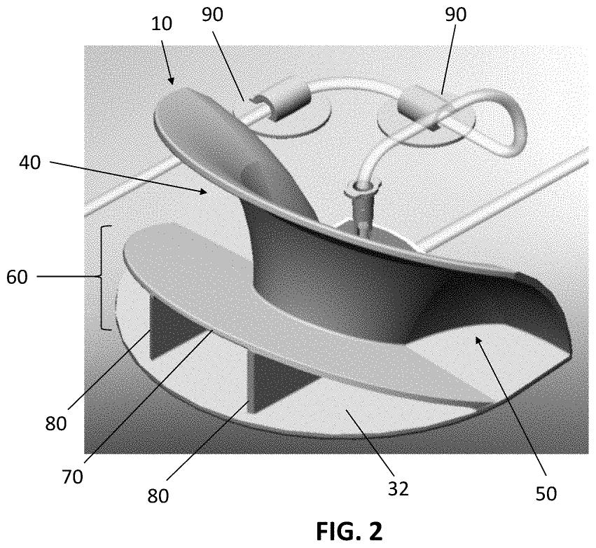

FIGS. 1 and 2 illustrate an embodiment of a medical port locator 10. As generally illustrated, a medical port locator 10 may be configured to surround and/or grip a separate device or component, such as a medical port 100 (which may be implanted partially or completely in a patient). Among other things, such a medical port locator may be used to stabilize a medical port—for example, in connection with a procedure.

A medical port locator, such as generally illustrated in FIGS. 1 and 2 may be configured to have a front (port-facing) side (see, e.g., FIG. 1), with a receiving/recessed portion 20, and a rear (user-facing) side (see, e.g., FIG. 2).

With respect to a front side of a medical port locator 10, the medical port locator 10 may include a receiving/recessed portion 20 (see, e.g., FIG. 1) that can be configured to have a size and shape to generally surround, grip, and/or engage at least a portion of a medical port 100 (which may have a smaller size and at least a portion with corresponding general shape). As generally illustrated in FIGS. 1 and 2, a medical port locator 10 may have an overall generally semi-circular shape (including a generally semi-circular (or C-shaped) receiving/recessed portion 20), and may be configured to substantially match up with a specific type of medical port 100.

As generally illustrated, a medical port locator 10 may also include an upper portion (or upper guard portion) 30, which may be contiguous with the receiving/recessed portion 20, and may extend so as to protect or shield a portion of a user's hand from a medical port or other devices used in connection with a medical port. For example and without limitation, a medical port locator may, among other things, protect a user's hand from needle pokes while facilitating the locating of a medical port (such as for an infusion).

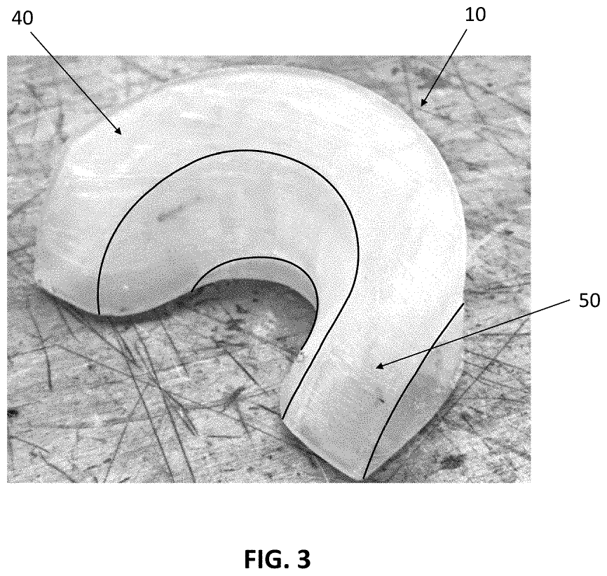

In embodiments, a rear side of a medical port locator 10 (see, e.g., FIG. 2) may include a base portion 32, and the rear side of the medical portion locator 10 may be configured to accept or conform to a portion of a user's hand (e.g., a portion corresponding to a thumb and index finger). With embodiments, a medical port locator 10 may have a first portion 40 and a second portion 50. In embodiments, the first portion and second portion may be sized, shaped, and/or otherwise configured differently. For example, in an embodiment, a first portion 40 may be configured to cover a portion of at least one finger of a user (e.g., a thumb) and a second portion 50 may be configured to cover a portion of at least one other finger of a user (e.g. forefinger or index finger). That is, with embodiments one portion of the medical port locator 10 may be sized larger than another portion—i.e., a “thumb” side (first portion 40) may be larger and/or longer than an “index finger” side (second portion 50). Additionally, a curved portion of the medical port locator associated with a receiving/recessed portion 20 may have a configuration that generally conforms to or with a portion of a user's hand between a thumb and index finger. In that regard, the embodiment generally illustrated in FIGS. 1 and 2 may be said to be a “right-handed” embodiment of a medical port locator 10. However, the configuration can be provided in a reverse or mirror image form so as to provide a “left-handed” embodiment (see, e.g., FIG. 3). Moreover, different sizes of medical port locators (e.g., small, medium, large, extra-large, etc.) can be provided to accommodate different user hand sizes.

With reference to FIG. 2, the rear side of a medical port locator 10 may include one or more additional structures. For example, a medical port locator 10 may include a back-side support structure 60 that may, among other things, provide support to portions of the medical port locator 10. In an embodiment, such as generally illustrated, a back-side support structure may include an angled portion 70 and one or more supports or lattice supports 80, which may support a portion of the medical port locator, such as an angled portion 70. With such an embodiment, a portion of a user's hand may rest upon a portion of the angled portion 70—which may, inter alia, provide a slope and/or help to direct a downward force (which may be more in line with the associated angle) with use of the medical port locator. Such a downward force may, for example, help direct additional force toward an area on a patient in which a medical port may be present. Alternatively or additionally, a lower portion of the receiving/recessed portion may have an inward slope (radially) which may help create a “pinch” around a medical port to be located.

In embodiments, the medical port locator may be comprised of plastic. The plastic may be configured to be somewhat rigid in form but yet still permit a given or desired amount of flexing in connection with at least portions of the medical port locator. By way of example and without limitation, a medical port locator 10 may be comprised of polypropylene. However, a medical port locator 10 may be comprised of other or additional materials, and may include non-plastic materials. Additionally, portions of the medical port locator 10 may include different textures, which may facilitate, for example, gripping. In embodiments, a medical port locator may be manufactured using one or more processes used in the manufacture of plastic components including, without limitation, injection molding, compression molding, and/or other molding processes.

With reference to FIGS. 1 and 2, a medical port locator system, which may comprise a medical port locator 10 and a medical port 100, may additionally include one or more anchors 90. In embodiments, such anchors may, for example and without limitation, be used to help secure tube or catheter, and may assist with removal of a device from a medical port (which may or may not be additionally held by a medical port locator). In embodiments, the underside of an anchor (e.g., the side opposite that associated with a line) may include an adhesive that may be temporarily used to secure an anchor 90 to a patient (e.g., disposable adhesive patches such as those used in connection with an electrocardiogram or an ECG). Such anchors may for example, be used in connection with various indwelling lines, such as peripheral IVs, PICC lines, and others—with or without a port locator device.

Various embodiments are described herein for various apparatuses, systems, and/or methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments.

Reference throughout the specification to “various embodiments,” “with embodiments,” “in embodiments,” or “an embodiment,” or the like, means that a particular feature, structure, or characteristic described in connection with the embodiment is included in at least one embodiment. Thus, appearances of the phrases “in various embodiments,” “with embodiments,” “in embodiments,” or “an embodiment,” or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment/example may be combined, in whole or in part, with the features, structures, functions, and/or characteristics of one or more other embodiments/examples without limitation given that such combination is not illogical or non-functional. Moreover, many modifications may be made to adapt a particular situation or material to the teachings of the present disclosure without departing from the scope thereof.

It should be understood that references to a single element are not necessarily so limited and may include one or more of such element. Any directional references (e.g., plus, minus, upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of embodiments.

Joinder references (e.g., attached, coupled, connected, and the like) are to be construed broadly and may include intermediate members between a connection of elements and relative movement between elements. As such, joinder references do not necessarily imply that two elements are directly connected/coupled and in fixed relation to each other. The use of “e.g.” in the specification is to be construed broadly and is used to provide non-limiting examples of embodiments of the disclosure, and the disclosure is not limited to such examples. Uses of “and” and “or” are to be construed broadly (e.g., to be treated as “and/or”). For example and without limitation, uses of “and” do not necessarily require all elements or features listed, and uses of “or” are intended to be inclusive unless such a construction would be illogical.

While processes, systems, and methods may be described herein in connection with one or more steps in a particular sequence, it should be understood that such methods may be practiced with the steps in a different order, with certain steps performed simultaneously, with additional steps, and/or with certain described steps omitted.

It is intended that all matter contained in the above description or shown in the accompanying drawings shall be interpreted as illustrative only and not limiting. Changes in detail or structure may be made without departing from the present disclosure.

What is claimed is:

1. A medical port locator, comprising:
a front side including a receiving portion and an upper portion, the receiving portion configured to engage at least a portion of a separate medical device; and
a rear side, the rear side configured to receive at least a portion of a hand;
wherein the medical port locator has a first portion configured to cover a portion of at least one finger of said hand and a second portion configured to cover a portion of at least another finger of said hand.

2. The medical port locator of claim 1, wherein the rear side includes a base portion.

3. The medical port locator of claim 1, wherein the rear side includes a base portion and an angled portion.

4. The medical port locator of claim 3, wherein the base portion and the angled portion are each substantially planar.

5. The medical port locator of claim 3, wherein the angled portion is disposed at a non-zero angle with respect to the base portion.

6. The medical port locator of claim 1, wherein the rear side includes one or more supports.

7. The medical port locator of claim 3, wherein one or more supports are provided between the base portion and the angled portion.

8. The medical port locator of claim 7, wherein the one or more supports are substantially perpendicular to the base portion.

9. The medical port locator of claim 3, wherein the angled portion is configured to direct a downward force toward the receiving portion.

10. The medical port locator of claim 1, wherein the first portion and the second portion have different shapes.

11. The medical port locator of claim 1, wherein one or the first portion and the second portion is larger than the other of the first portion and the second portion.

12. The medical port locator of claim 1, wherein one of the first portion and the second portion is configured to accommodate a thumb, and the other of the first portion and the second portion is configured to accommodate a different finger.

13. The medical port locator of claim 1, wherein a curved portion of the receiving portion is shaped to substantially conform to a portion of a hand between a thumb and an index finger.

14. The medical port locator of claim 1, wherein the medical port locator has a generally semi-circular shape.

15. The medical port locator of claim 1, including an anchor member; wherein the anchor member is configured to at least partially limit movement of a conduit connected to said separate medical device.

16. The medical port locator of claim 15, wherein the anchor member includes a first portion and a second portion; the first portion is substantially planar; and the second portion is substantially curved and configured to receive at least a portion of said conduit.

17. The medical port locator of claim 16, wherein an outer surface of the first portion of the anchor member includes an adhesive material.

18. The medical port locator of claim 1, wherein the receiving portion includes a recess configured to receive at least a portion of said separate medical device.
